(19) United States
(12) Patent Application Publication
(10) Pub. No.: US 2023/0041401 A1
(43) Pub. Date: Feb. 9, 2023

(54) COMMUNICATION BOOTH

(71) Applicant: OXTI CORPORATION, New Taipei City (TW)

(72) Inventor: Chih-Feng Ho, New Taipei City (TW)

(21) Appl. No.: 17/394,412

(22) Filed: Aug. 5, 2021

(51) Int. Cl. E04H 1/12; A61L 2/24; A61L 2/10; G08B 7/06

(52) U.S. Cl. CPC E04H 1/125; A61L 2/24; A61L 2/10; G08B 7/06; A61L 2202/14; A61L 2202/25

(57) ABSTRACT

The communication booth includes a booth body, a wireless network module, a charger module, a germicidal lamp, a control module, and at least a sensor. The booth body includes a booth enclosure and a booth door configured to a lateral side of the booth enclosure. The wireless network module, the charger module, and the germicidal lamp are configured inside the booth enclosure. The control module is electrically connected to the germicidal lamp and the sensor. The communication booth may provide wireless network service through the wireless network module, and charging service through the charger module. The control module controls the germicidal lamp to disinfect and sterilize the booth body. The sensor may detect activities inside or around the booth body. The control module, based on the detection from the sensor, turns off the germicidal lamp so as to avoid harms to people.

### BACKGROUND OF THE INVENTION

### (a) Technical Field of the Invention

The present invention is generally related to a communication booth, and more particular to a communication booth capable of epidemic prevention.

### (b) Description of the Prior Art

Conventional telephone booths are becoming obsolete due to the ubiquitous mobile phones. Therefore, conventional telephone booths are enhanced to multi-functional communication booths as disclosed, for example, in China Utility Model Patent No. CN212671320U.

However, during the current pandemic, these communication booths, with their closed spaces, may become a hotbed for virus and a breach to epidemic control.

### SUMMARY OF THE INVENTION

A novel communication booth is disclosed herein, which includes a booth body, a wireless network module, a charger module, a germicidal lamp, a control module, and at least a sensor. The booth body includes a booth enclosure and a booth door. The booth door is configured to a lateral side of the booth enclosure. The wireless network module is configured inside the booth enclosure. The charger module is configured inside the booth enclosure. the germicidal lamp is configured inside the booth enclosure. The control module and the germicidal lamp are electrically connected. The at least a sensor and the control module are electrically connected.

Preferably, a remote-control reception module is provided and electrically connected to the control module

Preferably, a door handle is configured on the booth door, and a sensor is configured on the door handle.

Preferably, an alarm issuing visual or audible alerts during disinfection and sterilization process of the communication booth is configured on the booth body.

Preferably, a desk or a chair is housed inside the booth body.

As described above, the communication booth may provide wireless network service through the wireless network module, and charging service through the charger module. The control module controls the germicidal lamp to disinfect and sterilize the booth body. In addition, the sensors may detect activities inside or around the booth body. The control module, based on the detection from the sensors, turns off the germicidal lamp so as to avoid harms to people, thereby achieving epidemic prevention. Additionally, a sensor may be configured on the door handle so that, when a person touches the door handle, the control module turns off the germicidal lamp. The alarm is for providing visual or audible alerts during the disinfection and sterilization process.

The foregoing objectives and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a perspective diagram showing a communication booth according to an embodiment of the present invention when its booth door is opened.

FIG. 2 is a perspective diagram showing the communication booth of FIG. 1 when its booth door is closed.

FIG. 3 is a sectional diagram showing the inside of the communication booth of FIG. 1.

FIG. 4 is a schematic diagram showing a germicidal lamp of the communication booth of FIG. 1 remotely controlled by a mobile phone.

FIG. 5 is a schematic diagram showing a germicidal lamp of the communication booth of FIG. 1 being automatically turned off when a person is close by or touching a booth body of the communication booth.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

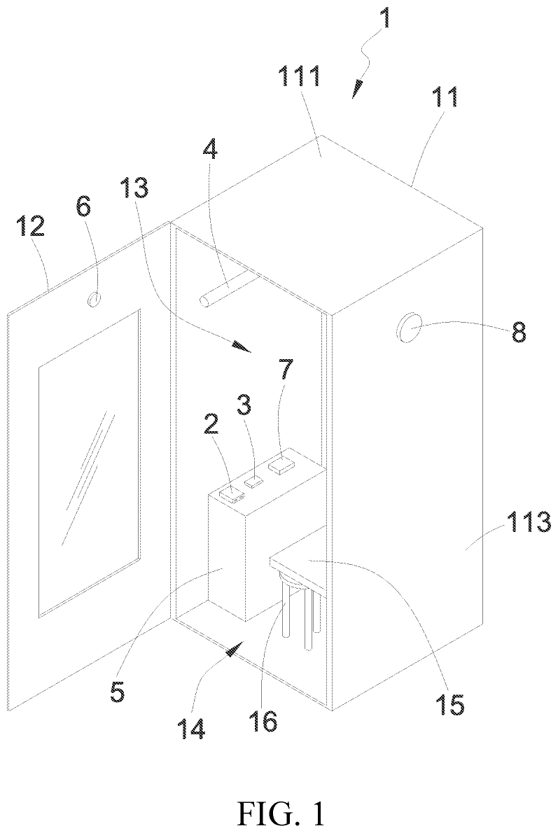

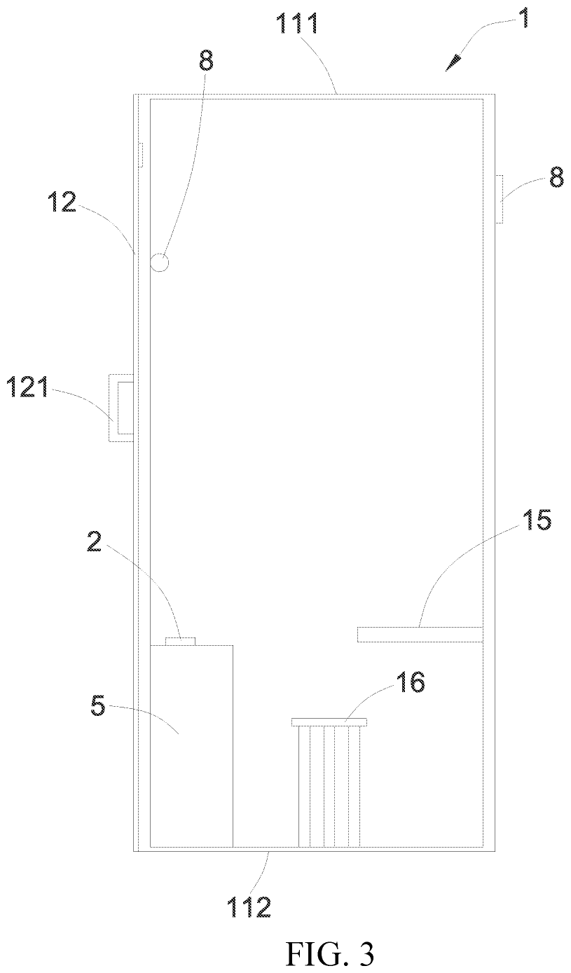

As shown in FIGS. 1 and 3, a communication booth according to an embodiment of the present invention includes a booth body 1, a wireless network module 2, a charger module 3, a germicidal lamp 4, a control module 5, and at least a sensor 6.

The booth body 1 includes a booth enclosure 11 and a booth door 12. The booth door 12 is hinged to a side of the booth enclosure 11. Specifically, the booth enclosure 11 includes a top side 111, a bottom side 112, and a number of lateral sides 113. The lateral sides 113 are disposed between the top side 111 and the bottom side 112, and the lateral sides 113 are edge-to-edge joined together to surround an internal space 13 with an opening 14. People may enter into or exit from the internal space 13 through the opening 14. The booth door 12 is for sealing the opening 14. A desk 15 or a chair 16 may be housed inside the booth body 1.

The wireless network module 2 is configured inside the booth enclosure 11. The wireless network module 2 is for providing wireless network service inside and around the booth body 1. Specifically, the wireless network module 2 may be a wireless router or a wireless access point (WAP). The wireless router (or wireless access point) is connected to the Internet through a cable and provides wireless communications. Computers, mobile phones, or tablets may link with the wireless router (or wireless access point) in a wireless manner and then with the Internet through the wireless router (or wireless access point). In other words, the wireless router and wireless access point provides wireless Internet service to computers, mobile phones, tablets inside or around the booth body 1.

The charger module 3 is configured inside the booth enclosure 11. The charger module 3 is for providing charging service. Specifically, the charger module 3 may be a wireless charger, a Universal Serial Bus (USB) charger, or other electrical charger.

The germicidal lamp 4 is configured inside the booth body 1. The germicidal lamp 4 is for providing disinfecting or sterilizing light, such as ultraviolet light, visible light, or infrared light, preferably ultraviolet light. The germicidal lamp 4 may be an ultraviolet light (UV) lamp, a metal halide lamp, a xenon lamp, a mercury lamp, or a fluorescent lamp. There may be multiple germicidal lamps 4 configured at various places inside the booth body 1.

The control module 5 and the germicidal lamp 4 are electrically connected. The control module 5 turns the germicidal lamp 4 on and off. The control module 5 at least includes a controller. The controller may be a central processing unit (CPU) or a microprocessor unit (MPU), and it is not limited as such. The control module 5 may further include a timer (not shown) so that the control module 5 can turn on and off the germicidal lamp 4 at scheduled times to provide automated disinfection and sterilization. The specific times and durations of the germicidal lamp 4's turning on and off can be set jointly by the controller and the timer.

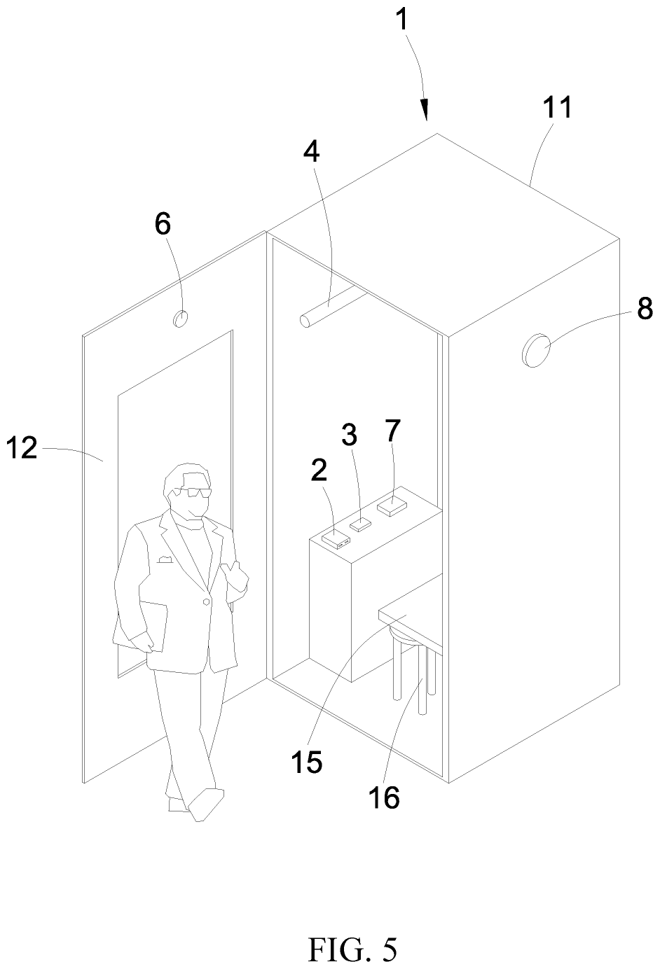

The one or more sensors 6 and the control module 5 are electrically connected. One of the sensors 6 may be a proximity detector for sensing whether a person is around or inside the booth body 1. The control module 5, based on the detection by the sensor 6 regarding whether someone is close by or touches the booth body 1. If yes, the control module 5 automatically turns off the germicidal lamp 4 to prevent the light from the germicidal lamp 4 to cause harm to the person. The sensors 6 may also include a light sensor, a pressure sensor, or a temperature sensor, but they are not limited as such. The light sensor detects light by light reflection or light blocking, the pressure sensor senses pressure change, and the temperature sensor detects temperature variation. As shown in FIGS. 3 and 5, for example, the light sensor may be a laser sensor or an infrared light sensor and emits a visible light or non-visible light to detects whether some object blocks the light along a specific direction. If yes, the light sensor notifies the controller of the control module 5. The pressure sensor may be configured beneath a carpet or mat in front of the booth door 12. When someone steps on the pressure sensor, the pressure sensor notifies the controller of the control module 5. The temperature sensor may be an infrared light temperature sensor to sense ambient temperature around the booth body 1. When the temperature sensor detects someone's body temperature, it notifies the controller of the control module 5. Furthermore, a door handle 121 may be configured on the booth door 12. A sensor 6 may be a touch sensor configured on the door handle 121. When the door handle 121 is touched by someone, the sensor 6 notifies the controller of the control module 5. After being notified by the sensor 6, the control module 5 automatically turns off the germicidal lamp 4 to prevent the light from the germicidal lamp 4 to cause harm to the person.

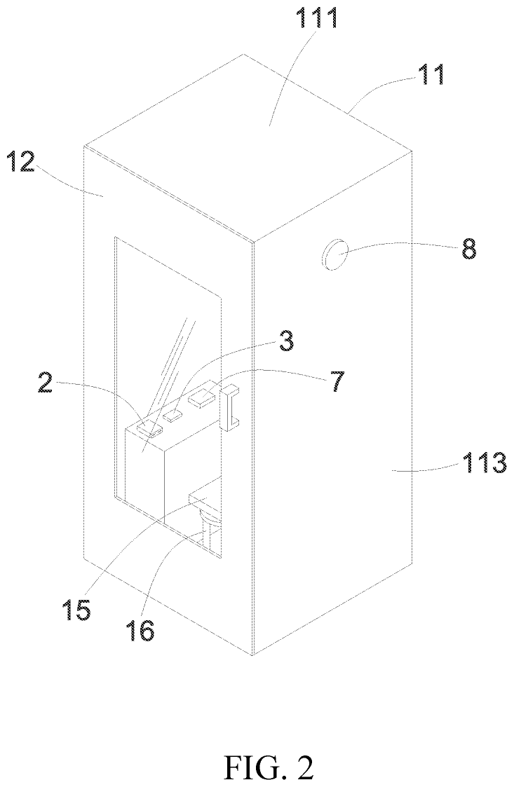

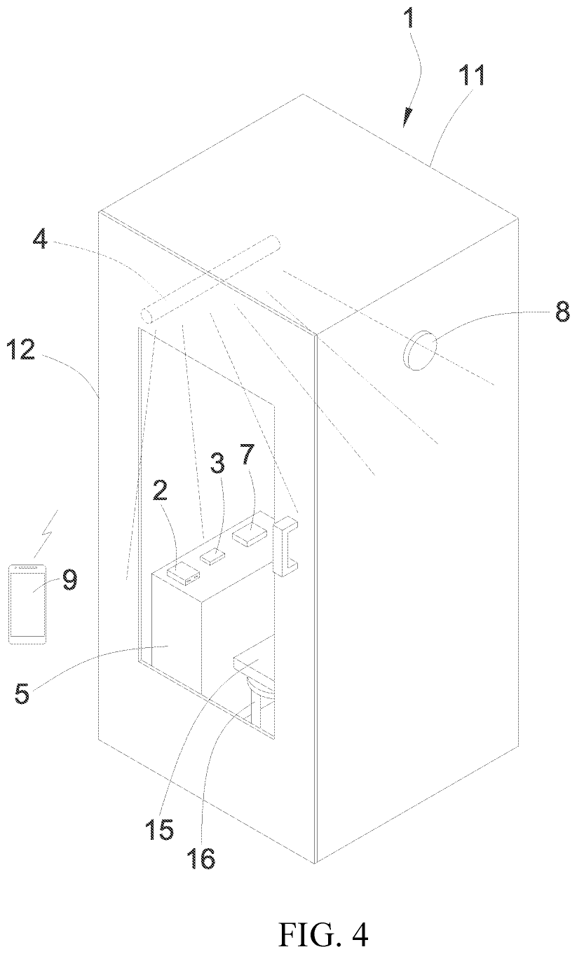

As shown in FIGS. 2 and 4, the communication booth may further include a reception module 7 electrically connected to the control module 5. The reception module 7 receives a remote signal, and relays the remote signal to the control module 5. The control module 5, according to the remote signal, control the germicidal lamp 4, such as turning on or off the germicidal lamp 4, or the duration of the germicidal lamp 4's illumination. A user may produce the remote signal through an application (APP) of a mobile phone 9 or a web browser and the remote signal is sent to the reception module 7 via Internet or other means. The reception module 7 relays the remote signal to the controller of the control module 5. The controller controls the germicidal lamp 4 to perform operations accordingly. The booth body 1 may further include an alarm 8 electrically connected to the control module 5. The control module 5 may activate the alarm 8 during the disinfection and sterilization process to issue visual (light or text) or audible alert.

As described above, the communication booth may provide wireless network service through the wireless network module 2, and charging service through the charger module 3. The control module 5 controls the germicidal lamp 4 to disinfect and sterilize the booth body 1. In addition, the sensors 6 may detect activities inside or around the booth body 1. The control module 5, based on the detection from the sensors 6, turns off the germicidal lamp 4 so as to avoid harms to people, thereby achieving epidemic prevention. Additionally, a sensor 6 may be configured on the door handle 121 so that, when a person touches the door handle 121, the control module 5 turns off the germicidal lamp 4. The alarm 8 is for providing visual or audible alerts during the disinfection and sterilization process.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the claims of the present invention.

I claim:

1. A communication booth, comprising:
a booth body comprising a hollow booth enclosure and a booth door configured to a lateral side of the booth enclosure;
a wireless network module configured inside the booth enclosure;
a charger module configured inside the booth enclosure;
a control module configured inside the booth enclosure;
a germicidal lamp configured inside the booth body electrically connected to the control module; and
at least a sensor electrically connected to the control module.

2. The communication booth according to claim 1, further comprising a reception module electrically connected to the control module.

3. The communication booth according to claim 1, wherein a door handle is configured on the booth door; and a sensor is configured on the door handle.

4. The communication booth according to claim 1, further comprising an alarm configured on the booth body issuing visual or audible alerts during disinfection and sterilization process of the communication booth.

5. The communication booth according to claim 1, further comprising a desk or a chair housed inside the booth enclosure.
